(19) United States
(12) Patent Application Publication
(10) Pub. No.: US 2026/0007590 A1
(43) Pub. Date: Jan. 8, 2026

(54) MOISTURE-LOCKING AND REPAIRING COMPOSITION FOR HAIR AND USE THEREOF

(71) Applicant: BLOOMAGE BIOTECHNOLOGY CORPORATION LIMITED, Shandong (CN)

(72) Inventors: Wenjie QU, Shandong (CN); Guixin XU, Shandong (CN); Songyan ZOU, Shandong (CN); Li LV, Shandong (CN)

(73) Assignee: BLOOMAGE BIOTECHNOLOGY CORPORATION LIMITED, Shandong (CN)

(21) Appl. No.: 18/993,723

(22) PCT Filed: Jul. 12, 2023
(86) PCT No.: PCT/CN2023/106944

§ 371 Date: Jan. 13, 2025

(30) Foreign Application Priority Data
Jul. 13, 2022 (CN) 202210820950.3

(51) Int. Cl. A61K 8/9728; A61K 8/73; A61Q 5/00

(52) U.S. Cl. CPC A61K 8/9728; A61K 8/735; A61Q 5/002; A61K 2800/85

(57) ABSTRACT

A moisture-locking and repairing composition for hair and the use thereof. The functional components of the composition are a yeast fermentation product filtrate or lysate, and a composite hyaluronic acid or a salt thereof, wherein the content of the yeast fermentation product filtrate or lysate is 1-90 wt %, and the content of the composite hyaluronic acid or a salt thereof is 1-20 wt %. Compared with a single component, the composition has a synergistic effect in the aspects of improving the strength and toughness of hair, preventing hair breakage, resisting frizz, etc., and has wide application prospects in the field of hair washing and hair care.

### TECHNICAL FIELD

The application relates to a moisture-locking and repairing composition for hair and use thereof, in particular to a moisture-locking and repairing composition for hair with the functions of improving the strength of hair, preventing hair breakage and resisting frizz and use thereof; and it belongs to the technical field of hair products.

### BACKGROUND ART

Nowadays, people have an increasing demand for hairdressing, thus the frequency of hair washing, perming, dyeing and styling is high. In addition, environmental stress can easily cause hair damage. These factors often lead to hair problems such as dryness and frizz, decreased smoothness, damaged hair quality, and easy breakage. Therefore, the development of active ingredients and products with hair conditioning and repair effects has become a research hotspot. At present, there are three types of hair repair active ingredients that are most widely used: 1) cationic polymer conditioners, such as polyquaternium series and cationic modified polysaccharides, are commonly used hair conditioners for improving the softness of hair and repairing surface hair scales; 2) protein, hydrolyzed protein and polypeptide macromolecules, which have the effects of repairing damaged hair, improving the mechanical strength of hair, and reducing hair breakage; 3) small molecule moisturizing ingredients, such as panthenol, amino acids, and small molecule organic acids have also been proven to have the effect of locking in moisture and moisturizing for hair and improving hair quality.

Hyaluronic acid (HA) is a macromolecular bioactive substance, which can bind a large number of water molecules through hydrogen bonds, and has the effect of moisturizing and locking in moisture. In recent years, it has been widely used in skin care products. China's technology for producing hyaluronic acid by microbial fermentation is in a leading position in the world. In recent years, local enterprises have taken the lead in producing oligomeric hyaluronic acid by enzymatic cleavage technology, and have expanded the deep moisturizing, anti-inflammatory, soothing, and repairing effects of hyaluronic acid of different molecular weights. However, up to now, the application of hyaluronic acid, especially oligomeric hyaluronic acid, in the field of hair care technology is still relatively rare.

### SUMMARY

The object of the present application is to provide a moisture-locking and repairing composition for hair, which is prepared by combining a yeast fermentation product filtrate or lysate and a composite hyaluronic acid or a salt thereof. During the experiment it was unexpectedly found that, the composition has a synergistic effect in improving hair strength, preventing hair breakage and resisting frizz as compared with a single component, thus it has broad application prospects in the field of hair washing and hair care.

The application provides a moisture-locking and repairing composition for hair, wherein its functional components are yeast fermentation product filtrate or lysate and a composite hyaluronic acid or a salt thereof, the content of the yeast fermentation product filtrate or lysate is 1-90 wt %, the content of the composite hyaluronic acid or a salt thereof is 1-20 wt %, and the total content of the two is less than or equal to 100 wt %.

Furthermore, in the above moisture-locking and repairing composition for hair, the content of the yeast fermentation product filtrate or lysate is preferably 5-50 wt %, more preferably 5-30 wt %.

Furthermore, in the above moisture-locking and repairing composition for hair, the content of the composite hyaluronic acid or a salt thereof is 1-15 wt %, more preferably 1-10 wt %.

For example, the content of the yeast fermentation product filtrate or lysate can be 1 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or 90 wt % etc. The content of the composite hyaluronic acid or a salt thereof can be 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt %, etc.

Furthermore, the ratio of content of the yeast fermentation product filtrate or lysate to the composite hyaluronic acid or a salt thereof is 1:4 to 9:1, preferably 1:3 to 2:1.

For example, the ratio of content of the yeast fermentation product filtrate or lysate to the composite hyaluronic acid or a salt thereof can be 1:4, 1:3.5, 1:3, 1:2.5, 1:2, 1:1.5, 1:1, 1.5:1, 2:1, 3:1, 4:1, 5:1, 6:1, 7:1, 8:1, or 9:1, etc.

Furthermore, the moisture-locking and repairing composition for hair may contain water as a solvent in addition to the functional components, for making up the balance of 100 wt %.

Furthermore, the yeast fermentation product filtrate or lysate according to the present application refers to the fermentation filtrate or lysate obtained by fermenting rice, bean or wheat and other plants with yeast. For example, the fermentation filtrate is a fermentation product obtained by propagating and/or fermenting yeast in a culture medium containing rice, bean or wheat, etc. The fermentation product filtrate is obtained by filtering the fermentation product, and the substance in the bacteria obtained by crushing the bacterial cells in the fermentation product is the fermentation product lysate. In addition to rice, bean or wheat, the culture medium may also comprise one or more ingredients necessary for fermentation, such as carbon source (sugar), nitrogen source (yeast powder, peptone, etc.), inorganic salt, etc., according to the needs of fermentation.

Preferably, the yeast fermentation product filtrate or lysate is a fermentation filtrate or lysate obtained by fermenting rice, especially brown rice (whole grain rice), with yeast, which may be called yeast/rice fermentation product filtrate or yeast/rice fermentation lysate. The yeast/rice fermentation product filtrate is prepared by the following method: brown rice is germinated and crushed into brown rice flour, which is then made into rice milk to be enzymatically hydrolyzed, and then yeast was added for fermentation, and the fermentation liquid is purified and sterilized to obtain the fermentation product filtrate.

Furthermore, the composite hyaluronic acid or a salt thereof is a composition of oligomeric hyaluronic acid or a salt thereof and low molecular weight hyaluronic acid or a salt thereof, wherein the molecular weight of the oligomeric hyaluronic acid or a salt thereof is less than or equal to 8 kDa, preferably less than or equal to 5 kDa, and the molecular weight of the low molecular weight hyaluronic acid or a salt thereof is 20-80 kDa, preferably 30-50 kDa.

Furthermore, the mass ratio of the oligomeric hyaluronic acid or a salt thereof to the low molecular weight hyaluronic acid or a salt thereof is 1:5 to 5:1, preferably 1:4 to 3:2.

For example, the mass ratio of the oligomeric hyaluronic acid or a salt thereof to the low molecular weight hyaluronic acid or a salt thereof can be 1:5, 1:4.5, 1:4, 1:3.5, 1:3, 1:2.5, 1:2, 1:1.5, 1:1, 1.5:1, 2:1, 3:1, 4:1, or 5:1, etc.

Furthermore, a salt of oligomeric or low molecular weight hyaluronic acid includes sodium salt, potassium salt, calcium salt, zinc salt, magnesium salt etc.

The moisture-locking and repairing composition for hair of the present application has unexpected synergistic effects in improving hair strength, preventing hair breakage and resisting frizz, and has a good application prospect in hair products. The hair product may be a hair care product or a hair cleansing product, and the hair product may be either a rinse-off or a leave-on product.

The present application also provides a hair product, wherein its functional components comprise the above moisture-locking and repairing composition for hair according to the present application.

Furthermore, the above hair product, in addition to the moisture-locking and repairing composition for hair according to the present application, may also comprise other functional components, such as other components with moisturizing, hydrating, moisture-locking, repairing, cleaning, anti-dandruff, anti-hair loss and other functions, for example, polypeptide, panthenol, amino acid, etc.

Furthermore, the content of the moisture-locking and repairing composition for hair in the hair product is 0.5-10 wt %, preferably 1-5 wt %, and more preferably 2-3 wt %.

For example, the content of the moisture-locking and repairing composition for hair in the hair product can be 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt%, 6.5 wt %, 7 wt %, 7.5 wt %, 8 wt %, 8.5 wt %, 9 wt %, 9.5 wt %, or 10 wt % etc.

Furthermore, the hair product may be a hair care product or a hair cleansing product. The hair product may be either a rinse-off type or a leave-on type.

Furthermore, the hair product may be various types such as shampoo, conditioner, hair mask, spray, etc.

Furthermore, the hair product may also comprise auxiliary ingredients necessary for preparing different dosage forms. Each auxiliary ingredient may be selected from the prior art, and the preparation of the dosage form may also be carried out according to the methods of prior art.

The present application combines yeast fermentation product filtrate or lysate, oligomeric hyaluronic acid or a salt thereof, and low molecular weight hyaluronic acid or a salt thereof. The yeast fermentation product filtrate or lysate contains rich active substances such as amino acids, polypeptides, minerals, etc., which can penetrate deeply into the hair core and repair damaged chemical bonds and thus repair keratin. Hyaluronic acids with different molecular weights are compounded to achieve effective penetration and distribution in the hair, reach the effect of three-dimensional repair and moisture locking, strengthen the hydrogen bonding effect inside the hair, and realize the consolidation of the keratin structure, thereby effectively improving the toughness of the hair, repairing and/or protecting the problem of damaged mechanical properties of the hair caused by various damage pressures (including bleaching, perming and dyeing, pollution exposure, ultraviolet radiation, multiple washing, high temperature, etc.); in addition, the composition also has the effect of lubricating the hair surface and resisting curling and frizz, thereby improving the performance of hair care products and hair cleansing products.

### BRIEF DESCRIPTION OF THE DRAWINGS

FIG. 1 is a fluorescence micrograph of the penetration of fluorescein-labeled sodium hyaluronate into hair, A is 30-50 kDa sodium hyaluronate, and B is 1460 kDa high molecular weight sodium hyaluronate.

FIG. 2 shows the strength repairing effect of the composition according to the present application on damaged hair in various conditions and healthy hair.

FIG. 3 shows the anti-curling effect of the composition in Example 1 according to the present application on hair.

### DETAILED DESCRIPTION

In order to make the above objects, features and advantages of the present application more obvious and easy to understand, the particular embodiments of the present application are described in detail below in conjunction with specific examples and drawings.

In the following examples, the INCI name of yeast/rice fermentation product filtrate used is SACCHAROMYCES/RICE FERMENT FILTRATE, and the raw materials used are commercially available products with the trade name: brown rice fermentation filtrate (Bioyouth™-Brice). The Lactobacillus/rye flour fermentation product used is a commercially available product. All hyaluronic acid used is from Bloomage Biotech Co., Ltd.

### Test Example 1: Tensile Strength Test

Each of the raw materials were weighed according to the formula in Table 1 below to add into water, then mixing evenly to obtain a moisture-locking and repairing composition for hair.

TABLE 1

| | Yeast/rice | Oligomeric | Low molecular | |
|---|---|---|---|---|
| | fermentation | sodium | weight sodium | |
| | product filtrate (g) | hyaluronate | hyaluronate | Water (g) |
| Example 1 | 10 | 4 g, 3 k-5 kDa | 6 g, 30 k-50 kDa | up to 100 |
| Example 2 | 1 | 0.6 g, 3 k-5 kDa | 0.9 g, 30 k-50 kDa | up to 100 |
| Example 3 | 5 | 6 g, 3-5 kDa | 9 g, 30 k-50 kDa | up to 100 |
| Example 4 | 5 | 8 g, 3 k-5 kDa | 12 g, 30 k-50 kDa | up to 100 |
| Example 5 | 30 | 4 g, 3 k-5 kDa | 6 g, 30 k-50 kDa | up to 100 |
| Example 6 | 50 | 4 g, 3 k-5 kDa | 6 g, 30 k-50 kDa | up to 100 |
| Example 7 | 90 | 4 g, 3 k-5 kDa | 6 g, 30 k-50 kDa | up to 100 |
| Comparative | 10 | 10 g, 3 k-5 kDa | / | up to 100 |
| Example 1 | | | | |
| Comparative | 10 | / | 10 g, 30 k-50 kDa | up to 100 |
| Example 2 | | | | |
| Comparative | 10 (Commercial | 4 g, 3 k-5 kDa | 6 g, 30 k-50 kDa | up to 100 |
| Example 3 | Lactobacillus/rye | | | |
| | flour fermentation | | | |
| | product) | | | |
| Comparative | / | 8 g, 3 k-5 kDa | 12 g, 30 k-50 kDa | up to 100 |
| Example 4 | | | | |
| Comparative | 2 | / | 2 g (1460 kDa High | up to 100 |
| Example 5# | | | molecular weight | |
| | | | sodium hyaluronate) | |
| Blank control | / | / | / | 100 |

The tensile strength test of the moisture-locking and repairing compositions for hair prepared above was carried out, and the test method was as follows:

Firstly, the compositions prepared in the Examples and Comparative Examples were diluted with deionized water: the aqueous solutions of the compositions except for that of Example 2 were diluted with water, so that the content of active ingredients in the solution of each composition was 2.5wt %. The prepared solutions were respectively filled in spray bottles or pump head bottles to be used as test samples.

#For Comparative Example 5, due to the high viscosity of 1460 KDa sodium hyaluronate, it is impossible to prepare a 10% solution; thus a 2% solution was prepared, and the addition amount of yeast/rice fermentation product filtrate is also adjusted to 2%.

Thirteen severely damaged hair bundles (repeatedly bleached hair bundles, a length of 15 cm, a weight of 1 g) were selected, all of which were pre-washed with 10% SDS and naturally dried under constant temperature of (25±2)° C. and constant humidity of (50±5) %. After the hair bundles were completely dry, the method of applying a hair care spray was imitated, and the twelve hair bundles were sprayed with an equal amount (0.3 g) of the test samples of each Examples and Comparative Examples, and one hair bundle was sprayed with an equal amount (0.3 g) of clean water. After naturally drying under constant temperature and humidity conditions, the above operation was repeated for ten times. After the last application and drying, all hair bundles must be washed with deionized water to remove the residual samples on the surface of the hair bundles, then placing in a constant temperature and humidity environment to dry. The diameter of the hair was measured by using a SN-1200W high-definition camera. The specific method was to take the average of the diameters at three locations in the middle section. Thirty hair strands with a diameter difference within 10 μm were selected from each of the thirteen hair bundles for single fiber strength test by fiber strength tester. The tensile strength of the control group and each sample group was calculated and compared by the following formula:

σ=Fb/So

wherein σ is the tensile strength, Fb is the maximum force that the hair specimen withstands when it is broken, and So is the original cross-sectional area of the specimen. In addition, the average elastic modulus of the hair was calculated from the elastic modulus area of the tensile curve.

The results of the hair tensile strength test are shown in Table 2 below:

TABLE 2

| | | Significance analysis, |
|---|---|---|
| | | as compared |
| | Average tensile | with blank |
| | strength (MPa) | control group |
| Example 1 | 235.2 ± 25.3 | P < 0.001 |
| Example 2 | 232.8 ± 30.5 | P < 0.01 |
| Example 3 | 230.2 ± 26.4 | P < 0.01 |
| Example 4 | 225.9 ± 28.6 | P < 0.01 |
| Example 5 | 228.1 ± 28.3 | P < 0.01 |
| Example 6 | 226.5 ± 30.5 | P < 0.01 |
| Example 7 | 225.2 ± 29.4 | P < 0.01 |
| Comparative Example 1 | 212 ± 27.2 | P < 0.05 |
| Comparative Example 2 | 220.9 ± 35.6 | P < 0.01 |
| Comparative Example 3 | 224.8 ± 28.6 | P < 0.01 |
| Comparative Example 4 | 224.3 ± 27.4 | P < 0.01 |
| Comparative Example 5 | 193.4 ± 29.8 | P > 0.1 |
| Blank control | 186.4 ± 23.3 | / |

It can be seen from the results in the above Table 2 that, the tensile strength of the hair specimens treated by compositions from Examples 1-7 is higher than that of the hair specimens of the blank control, indicating that the combination of yeast/rice fermentation product filtrate and sodium hyaluronate is indeed beneficial to improving the strength of hair.

The tensile strength of the hair specimens treated by composition from Example 1 is significantly higher than that of the hair specimens of Comparative Examples 1 and 2 and the blank control, indicating that the product of a combination of yeast/rice fermentation product filtrate and the two sodium hyaluronates with different molecular weights (i.e., oligomeric sodium hyaluronate, and low molecular weight sodium hyaluronate) is more conducive to improving the strength of hair than the product of a combination of yeast/rice fermentation filtrate and a single sodium hyaluronate, thereby proving the necessity of compounding the two sodium hyaluronates with different molecular weights. The tensile strength results of the hair specimens treated by compositions from Examples 1-7 indicate that, the closer the ratio of yeast/rice fermentation filtrate to the two sodium hyaluronates with different molecular weights is to 1:1, the more obvious the improvement in hair strength is.

At the same time, the tensile strength of the hair specimens treated by composition from Example 1 is also higher than that of the hair specimens treated by compositions from Comparative Examples 3-4, indicating that in the composition, the combination of the yeast/rice fermentation product filtrate and the two sodium hyaluronates has synergistic effects on improving the strength of hair; and if the yeast/rice fermentation product filtrate is replaced with other types or varieties of fermentation product filtrate, the composition will not have a significant synergistic effect. The tensile strengths of the hair specimens treated by compositions from Example 1, and Comparative Examples 1-2 are significantly higher than that of the hair specimens treated by composition from Comparative Example 5, indicating that the choice of the molecular weight of hyaluronic acid has a significant effect on improving hair strength.

From the results of the penetration experiment of fluorescein-labeled sodium hyaluronate in hair in FIG. 1, it can be seen that the sodium hyaluronate with a high molecular weight of 1460 kDa has no permeability, while the sodium hyaluronate with a molecular weight of 30-50 kDa can penetrate into the hair and even the hair core, thereby achieving a repairing effect. The specific experimental steps are as follows:

0.2 g of sodium hyaluronate was taken to add to 2 mL of 0.05 mol/L NaOH aqueous solution in a test tube with stopper, then sealing the tube with the stopper, and vortexing to dissolve the sodium hyaluronate; accurately adding 0.04 g of FITC and sealing with the stopper, mixing well with a vortex mixer and placing in a 95° C. water bath for constant temperature reaction for 45 min, then taking it out and cooling to room temperature; adding 18 mL of sodium chloride saturated anhydrous ethanol, then centrifuging and discarding the supernatant. The resulting precipitate is the crude sodium hyaluronate labeled with FITC fluorescence. 20 mL of sodium chloride saturated anhydrous ethanol was added to the precipitate to vortex to evenly disperse the labeled sodium hyaluronate precipitate in the sodium chloride saturated anhydrous ethanol, then centrifuging and discarding the upper alcohol wash solution, which is the first alcohol wash. After repeating the alcohol wash for 6 times, the precipitate was freeze-dried to obtain the FITC fluorescent labeled sodium hyaluronate product. Severely damaged hair was used for the experiment. 0.05 g of FITC fluorescent labeled sodium hyaluronate product was accurately weighed to add in a test tube with stopper, adding 10 mL of deionized water and sealing to vortex to dissolve it. Twenty hairs were randomly selected to immerse in the fluorescently labeled sodium hyaluronate solution; after soaking for 4 hours, they were taken out, and the surface of the hairs was thoroughly cleaned to remove the sodium hyaluronate remaining on the surface. The hair cross-sections were then obtained by cryosectioning to observe under fluorescence microscope. It can be seen from the figure that, the fluorescently labeled sodium hyaluronate with high molecular weight of 1460 kDa (FIG. 1, right panel) has little penetration into the hair, while the fluorescently labeled 30-50 kDa sodium hyaluronate (FIG. 1, left panel) can be observed to have significant fluorescence in the hair and even in the hair core.

Further analysis of the tensile curve characteristics obtained in each experiment shows that, the elastic modulus and elongation at break of hairs are different when different test samples are tested. The elastic modulus and elongation at break of hair of some test samples are shown in Table 3 below:

TABLE 3

| | Average elastic modulus of | Average elongation at |
|---|---|---|
| | hair (GPa) | break |
| Example 1 | 4.5 ± 0.78 (*) | 67.2% ± 6.6% (*) |
| Comparative | 4.23 ± 0.86 | 68.3% ± 4.3% (*) |
| Example 1 | | |
| Comparative | 4.58 ± 0.8 (*) | 64.1% ± 7.0% |
| Example 2 | | |
| Blank control | 4.11 ± 0.51 | 63.2% ± 4.8% |

Note:
(*) indicates that the statistical data of this group are significantly different from those of the blank control group (P < 0.05).

The above results indicate that, there are differences in the effects on the mechanical properties of hair between the compositions of yeast/rice fermentation product filtrate compounded with different hyaluronic acids. Particularly, when the yeast/rice fermentation product filtrate is compounded with oligomeric sodium hyaluronate (Comparative Example 1), the elongation at break of the hair may be significantly improved, while the elastic modulus of the hair is not greatly affected; when the yeast/rice fermentation product filtrate is compounded with low molecular weight sodium hyaluronate (Comparative Example 2), the elastic modulus of the hair may be significantly improved, while the elongation at break of the hair is not greatly affected; however, when the yeast/rice fermentation product filtrate is compounded with two kinds of sodium hyaluronate (i.e., oligomeric sodium hyaluronate, and low molecular weight sodium hyaluronate), the elastic modulus and elongation at break of the hair can be significantly improved at the same time (Example 1). Therefore, it is necessary to compound these two kinds of sodium hyaluronate in the composition. The principle is that low molecular weight sodium hyaluronate and oligomeric sodium hyaluronate have complementary effects in enhancing the strength of hair: low molecular weight sodium hyaluronate is more effective in improving the elastic modulus of hair, while oligomeric sodium hyaluronate is more effective in improving the elongation at break of hair. This may be because low molecular weight sodium hyaluronate has a strong moisture-locking effect, and dominates the strengthening of the hydrogen bond network; while oligomeric sodium hyaluronate has higher activity and permeability, and thus interacts more strongly with keratin.

### Test Example 2: Antioxidant Activity Test

The yeast/rice fermentation product filtrate is rich in active ingredients such as peptides, amino acids, anti-active oxygen phytic acid, ferulic acid, lactic acid, plant amides and sterols. In addition to having high permeability and the property of enhancing the ability of the composition to improve the moisture-locking and strength of hair, these small molecule active ingredients also have antioxidant and free radical scavenging activities. Since hair will generate oxidative free radicals under ultraviolet radiation, which will further damage the hair structure and cause the hair strength to decrease, thus the yeast/rice fermentation product filtrate in the composition will also endow the composition the effect of protecting the hair from ultraviolet damage. In order to verify the antioxidant property, the solutions of the moisture-locking and repairing composition for hair in Example 1, Comparative Example 3 and Comparative Example 4 were diluted with deionized water until the moisture-locking and repairing composition for hair was 2.5 wt %, and used as test samples. The hair treatment method is as follows: selecting healthy hair bundles to wash with 10% SDS, storing them under constant temperature and humidity conditions of (25±2)° C. and (50±5)%, then treating each hair bundle according to the method in Table 4; wherein the ultraviolet irradiation is carried out in a xenon lamp simulation box, after 24 hours of irradiation, spraying an equal amount of the sample on the hair bundle. The treated hair bundles were tested for the tensile strength according to the method of Test Example 1.

Determination of DPPH clearance rate: 2 mL of 2 mmol/L DPPH solution was taken as the blank group, 2 mL of anhydrous ethanol instead of DPPH solution was used in the control group; and in sample groups, 2 mL of anhydrous ethanol and 2 mL of each of the samples of Example 1, Comparative Example 3 and Comparative Example 4 to be tested were respectively diluted to a composition concentration of 2.5 wt %, shaking to mix well, reacting in the dark for 30 min, then measuring the absorbance value (D value) at 517 nm, repeating the above experiment 3 times. The clearance rate was calculated, and the antioxidant activity of each sample was analyzed. Clearance rate (%)=[1−(D of tested sample−D of blank group)/D of control group]×100%. The experimental results are shown in Table 4 below.

TABLE 4

| | | The percentage of | | |
|---|---|---|---|---|
| | | decrease of | | |
| | | average tensile | Significance analysis | |
| | | strength | of average tensile | |
| | | compared with | strength, as compared | |
| | | that of healthy | with data group of | |
| | | hair (without UV | healthy hair in the | DPPH |
| | Average tensile | irradiation) in | blank group after UV | clearance |
| | strength/MPa | blank group | irradiation | rate (%) |
| Hair treated with | 195.1 | −6.8% | P < 0.01 | 35% |
| sample of Example 1, | | | | |
| after UV irradiation | | | | |
| Hair treated with | 182.3 | −12.8% | No significant | 15% |
| sample of | | | difference | |
| Comparative | | | | |
| Example 3, after UV | | | | |
| irradiation | | | | |
| Hair treated with | 180.2 | −13.8% | No significant | 12% |
| sample of | | | difference | |
| Comparative | | | | |
| Example 4, after UV | | | | |
| irradiation | | | | |
| Healthy hair in the | 169.6 | −19.0% | / | / |
| blank group, after UV | | | | |
| irradiation | | | | |
| Healthy hair in the | 209.2 | / | / | / |
| blank group, without | | | | |
| UV irradiation | | | | |

It can be seen from the above data that, when healthy hair is not treated with any product, the strength (tensile strength) decreases by 19.0% after ultraviolet irradiation, while after healthy hair is treated with sample of the composition in Example 1, the degree of strength decrease after ultraviolet irradiation is significantly reduced, which is significantly less than that of Comparative Examples 3-4, demonstrating that the composition of Example 1 has ultraviolet protection performance for hair, and indicating that a combination of the yeast/rice fermentation product filtrate of the present application and the compounded hyaluronic acids has synergistic effect on antioxidant activity.

### Test Example 3: Combing Performance Test

The experiment was carried out at a temperature of (25±2)° C. and a humidity of (50±5)%. A real human hair piece of 40 cm in length and 25 g in weight was selected, fully moistening the hair piece with warm water at 40° C.; a dropper was used to absorb 5 mL of 10 wt % SDS aqueous solution to evenly apply it on the front and back of the hair piece, then rubbing to produce foam. When rubbing, hair folding in half or twisting together should be avoided to prevent the hair from tangling, then rinsing off the foam with warm water, repeating the above operation twice, and place the hair piece in a constant temperature and humidity environment to dry naturally.

The moisture-locking and repairing composition for hair of Example 1 and Comparative Examples 1-3 were diluted with deionized water until the moisture-locking and repairing compositions for hair were 2.5 wt %, to be used as test samples.

1.5 g of the test sample prepared in each example and comparative example was accurately weighed to spray evenly on the hair bundle, and the hair was simply combed with a comb, while clean water was used as a blank control. After the hair bundles were naturally dried in a constant temperature and humidity environment, a combing instrument was used to test the dry combing work of each hair bundle. 2 pieces of hair of the same specifications were used to perform the testing, repeating the testing seven times. The combing curve was obtained by data processing, and the combing work can be obtained by integrating the combing curve. By comparing the combing work before and after application of the test sample, the ability of the sample to improve the combability of the hair bundle can be obtained.

The experimental results are shown in Table 5 below:

TABLE 5
Dry combing work of hair (unit: *10 cN*mm)

| | | | | | Comparative |
|---|---|---|---|---|---|
| | Example 1 | | Blank control | | Example 1 |
| Testing | Before | After | Before | After | Before |
| times | application | application | application | application | application |
| 1 | 1056 | 810 | 1051 | 1037 | 1046 |
| 2 | 1122 | 837 | 1117 | 1099 | 1112 |
| 3 | 1224 | 870 | 1220 | 1205 | 1229 |
| 4 | 1233 | 942 | 1233 | 1229 | 1230 |
| 5 | 1335 | 945 | 1325 | 1319 | 1339 |
| 6 | 1353 | 957 | 1349 | 1342 | 1357 |
| 7 | 1359 | 987 | 1358 | 1355 | 1363 |

| | Comparative | Comparative | | Comparative | |
|---|---|---|---|---|---|
| | Example 1 | Example 2 | | Example 3 | |
| Testing | After | Before | After | Before | After |
| times | application | application | application | application | application |
| 1 | 964 | 1055 | 958 | 1052 | 858 |
| 2 | 982 | 1120 | 976 | 1126 | 876 |
| 3 | 1023 | 1220 | 999 | 1231 | 925 |
| 4 | 1087 | 1223 | 1057 | 1236 | 984 |
| 5 | 1098 | 1345 | 1088 | 1344 | 995 |
| 6 | 1178 | 1358 | 1193 | 1362 | 1083 |
| 7 | 1234 | 1367 | 1229 | 1363 | 1141 |

It can be seen from the results of Comparative Examples 1-2 and the blank control in the above table that, the combing work of hair can be reduced by using a single hyaluronic acid in combination with the yeast/rice fermentation product filtrate. From the comparison results between Example 1 and Comparative Examples 1-2, it can be seen that the effect of reducing hair combing work by using a combination of compounded hyaluronic acids and the yeast/rice fermentation filtrate is better than a combination of single molecular weight hyaluronic acid and the yeast/rice fermentation filtrate. Furthermore, it can be seen from the results of Example 1 and Comparative Example 3 that, the combing effect of a combination of compounded hyaluronic acids and the yeast/rice fermentation filtrate is better than a combination of compounded hyaluronic acid and other fermentation filtrates. This indicates that the composition according to the present application has an optimized technical effect of improving the combability of hair strands. This is because the composition according to the present application comprises active substances with different molecular weights, which can also form a dense and good lubricating film on the surface of the hair, thereby playing a role in lubricating the surface of the hair. Improvement of combability is very important for preventing hair breakage, because in daily life, hair is difficult to comb and easily tangled, which is an important reason for hair breakage during combing. Therefore, the composition according to the present application can not only improve the strength of single hair fiber, but also improve the combability of hair bundles, thereby achieving a comprehensive technical effect of preventing hair breakage.

### Test Example 4: Tensile Strength Test of Hair in Different Conditions

The damaged hair caused by multiple bleaching was used in the aforementioned experiments. However, in daily life, hair faces various pressure scenarios, such as hair dyeing, chemical perming, exposure to pollutants, ultraviolet radiation, multiple washing, thermal straightening, etc.

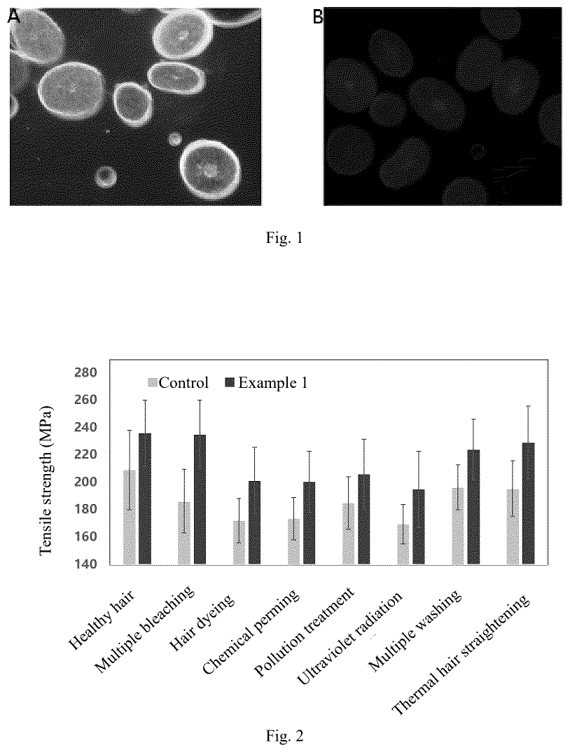

In order to test the change in hair strength when the composition in Example 1 according to the present application is applied to various types of damaged hair, the solution of the moisture-locking and repairing composition for hair in Example 1 was diluted with water to form a test sample with a concentration of 2.5 wt %. According to the method of Test Example 1, the test sample was applied on different damaged hairs respectively, so as to test the tensile strength of the hairs before and after application of the composition, and clean water was used as a blank control. The experimental results are shown in FIG. 2. It can be seen from FIG. 2 that, the composition of the present application can be used to repair the strength of various types of damaged hair, and can also improve the strength of healthy hair.

### Test Example 5: Anti-curling and Anti-frizz Performance

The moisture-locking and repairing composition for hair in Example 1 was diluted with water to prepare a test sample with a concentration of 2.5 wt %.

Anti-curling test: 6 hair bundles (3 healthy hair bundles, and 3 damaged hair bundles; all 15 cm in length, and all 1 g in weight) were pre-washed with 10% SDS, then naturally drying under constant temperature and humidity conditions of (25±2)° C. and (50±5)% humidity. After the hair bundles were completely dry, 0.3 g of deionized water and the test sample were sprayed on the six hair bundles respectively, drying naturally under constant temperature and humidity conditions, placing in an environment with a humidity of about 80% (simulating a rainy day), taking pictures at 0 h and 4 h respectively, and comparing the curliness and frizziness of the hair bundles in the high humidity environment and the changes in the volume of the hair bundles, so as to obtain the anti-frizz performance of the product.

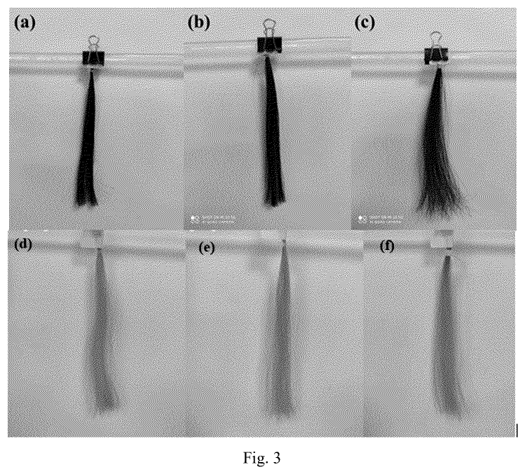

The experimental results are shown in FIG. 3. FIG. 3a is a healthy hair bundle before treatment in a high humidity environment (for comparison of before and after applying the composition). FIG. 3b is an image of a healthy hair bundle after being treated with a test sample, then leaving at 80% high humidity for 4 hours. FIG. 3c is an image of a healthy hair bundle after being treated with deionized water, then leaving at 80% high humidity for 4 hours. FIG. 3e is a damaged hair bundle before treatment in a high humidity environment (for comparison of before and after applying the composition). FIG. 3d is an image of the damaged hair bundle after treatment with a test sample, then leaving at 80% high humidity for 4 hours. FIG. 3f is an image of the damaged hair bundle after treatment with deionized water, then leaving at 80% high humidity for 4 hours. It can be seen from FIG. 3 that, the volume of the hair bundle not treated with the test sample increases significantly in a humid environment, and the hair bundle, especially the ends, becomes curly and frizzy, while the volume of the hair bundle treated with a test sample changes little in a high humidity environment. Therefore, the composition according to the present application also has anti-frizz and anti-curling effects on hair, which can prevent hair from becoming unmanageable under high humidity conditions.

### Test Example 6: Hair Moisture-Locking Efficacy Test

Hair contains both free water and bound water, wherein free water has a greater impact on the properties of hair. Too much free water often causes the destruction of hydrogen bonds in hair and the decrease of keratin crystallinity, thereby reducing strength. In the composition according to the present application, sodium hyaluronates with different molecular weights are combined with yeast/rice fermentation product filtrate, wherein the component with moisture-locking activity has a molecular weight ranging from tens of thousands to hundreds Dalton, and contains abundant moisturizing groups (including carboxyl, amino, hydroxyl), thereby producing a three-dimensional moisture-locking effect, strengthening the hydrogen bonding effect in the hair, and playing a role in consolidating the keratin structure, ultimately it reflects an improvement of the strength of hair.

The method for testing the hair moisture-locking effect is as follows: the experiment is carried out at a temperature of (25±2)° C. and a humidity of 50±5%. 6 bundles of human hair with a length of 40 cm and a mass of 25 g were taken to pre-wash with 10% SDS, then placing in a constant temperature and humidity environment for 24 hours to dry naturally. Firstly, a certain mass of hair bundles in each group was taken to place in the moisture analyzer for testing. The temperature is set to 65° C. and the test time is set to 20 minutes to test the water loss rate at 65° C. (reflecting the free water content in the hair, excluding structural water). Then, 1 g of deionized water and 1g of 0.5% hair moisture-locking repair composition dilution were accurately measured to spray evenly on the hair respectively, drying at a constant temperature and humidity for 12 hours, then rinsing the hair to remove the sample remaining on the surface of the hair. After repeating this operation for ten times, a certain amount of hair bundles were taken to place in a moisture analyzer to test the water loss rate at 65° C. The thermal weight loss of the hair during the process was recorded to obtain the free water content in the hair. The experimental results are shown in Table 6 below:

TABLE 6

| | | Significance analysis, as |
|---|---|---|
| | Free water content in hair | compared with blank |
| | (average value) | control group |
| Example 1 | 8.0%* | P < 0.01 |
| Comparative | 9.1% | P < 0.05 |
| Example 1 | | |
| Comparative | 8.8% | P < 0.05 |
| Example 2 | | |
| Comparative | 8.4%* | P < 0.05 |
| Example 3 | | |
| Comparative | 8.5%* | P < 0.05 |
| Example 4 | | |
| Comparative | 10.0% | P > 0.1 |
| Example 5 | | |
| Blank | 9.8% | / |
| control | | |

It can be seen from the results in the above table that, the composition of Example 1 has the most significant effect on reducing the free water content in hair, indicating that the composition of the present application has the best hair moisture-locking effect, and this moisture-locking effect is an important reason for improving the strength of hair and producing the anti-frizz and anti-curling technical effect.

### Test Example 7: Screening of the Content of the Hair Moisture-locking and Repairing Composition

The moisture-locking and repairing composition for hair in Example 1 was diluted with deionized water to make the content of the effective ingredients of the moisture-locking and repairing composition for hair as shown in Table 7 below. The resulting aqueous solutions of the moisture-locking and repairing composition for hair with different mass percentages were tested for hair tensile strength according to the method in Test Example 1. The results are shown in Table 7 below.

TABLE 7

| Content of the | | Increase ratio, | Significance |
|---|---|---|---|
| moisture-locking | Average | as | analysis, as |
| and repairing | tensile | compared with | compared with |
| composition | strength | blank control | blank control |
| for hair | (MPa) | group | group |
| 0 (Blank clean water control) | 186.4 | / | / |
| 0.1% | 190.1 | 2.0% | P > 0.1 |
| 0.5% | 198.3 | 6.4% | P < 0.05 |
| 1% | 214.2 | 14.9% | P < 0.001 |
| 2.5% | 235.2 | 26.2% | P < 0.001 |
| 5.0% | 235.0 | 26.1% | P < 0.001 |
| 10% | 233.4 | 25.2% | P < 0.001 |

The above results show that, in actual use scenarios, the composition does not show a significant effect of enhancing hair strength when added at a concentration of 0.1%, but it can show a significant effect of enhancing hair strength when added at a concentration of 0.5-10%. Therefore, preferably the composition is used in a hair product at a concentration of 0.5-10%, more preferably the composition is used in a hair product at a concentration of 1-5%, and still more preferably 2-3%.

### Test Example 8: Screening of the Compounding Ratio of Oligomeric Sodium Hyaluronate and Low Molecular Weight Sodium Hyaluronate

According to the method of Example 1, yeast/rice fermentation product filtrate, oligomeric sodium hyaluronate, low molecular weight sodium hyaluronate and water are mixed to prepare a solution of the moisture-locking and repairing composition for hair, wherein content of the yeast/rice fermentation product filtrate is 10 wt %, and the total content of the oligomeric sodium hyaluronate and the low molecular weight sodium hyaluronate is 10 wt %. By changing the mass ratio of oligomeric sodium hyaluronate and low molecular weight sodium hyaluronate, multiple groups of moisture-locking and repairing composition for hair solutions as shown in Table 8 below were obtained. Each group of moisture-locking and repairing composition for hair solution was diluted with water to obtain a test sample with a hair moisture-locking and repairing composition content of 2.5wt %. The hair tensile strength of a test sample was tested according to the method in Test Example 1. The test results are shown in Table 8 below:

TABLE 8

| | | | Mass ratio of | | |
|---|---|---|---|---|---|
| | Yeast/rice | Composite | oligomeric sodium | Average | Increase ratio, |
| | fermentation | sodium | hyaluronate to low | tensile | as compared |
| | product filtrate | hyaluronate | molecular weight | strength | with blank |
| No. | (wt %) | (wt %) | sodium hyaluronate | (MPa) | control group |
| 1 | 10 | 10 | 2:3 | 235.2 | 26.2% |
| 2 | 10 | 10 | 5:1 | 214.3 | 15.0% |
| 3 | 10 | 10 | 3:1 | 218.2 | 17.1% |
| 4 | 10 | 10 | 3:2 | 223.7 | 20.0% |
| 5 | 10 | 10 | 1:1 | 225.3 | 20.8% |
| 6 | 10 | 10 | 1:2 | 232.5 | 24.7% |
| 7 | 10 | 10 | 1:3 | 227.3 | 23.9% |
| 8 | 10 | 10 | 1:4 | 228.1 | 22.4% |
| 9 | 10 | 10 | 1:5 | 221.5 | 18.8% |
| 10 | 10 | 10 | 1:6 | 212.2 | 13.8% |
| 11 | 10 | 10 | 6:1 | 201.8 | 8.3% |
| Blank | / | / | / | 186.4 | / |
| control | | | | | |

The above experimental results show that, when the ratio of oligomeric sodium hyaluronate to low molecular weight sodium hyaluronate is in a range of 1:5 to 5:1, the hair strength can be effectively improved, and the composition shows a good synergistic effect. Particularly, when the ratio of oligomeric sodium hyaluronate to low molecular sodium hyaluronate is in a range of 3:2 to 1:4, the strength improvement ratio exceeds 20%, so the mass ratio of oligomeric sodium hyaluronate to low molecular sodium hyaluronate is preferably 3:2 to 1:4.

1. A moisture-locking and repairing composition for hair, wherein its functional components comprise a yeast fermentation product filtrate or lysate and a composite hyaluronic acid or a salt thereof, the content of the yeast fermentation product filtrate or lysate is 1-90 wt %, and the content of the composite hyaluronic acid or a salt thereof is 1-20 wt %.

2. The moisture-locking and repairing composition for hair according to claim 1, wherein the content of the yeast fermentation product filtrate or lysate is 5-50 wt %, and the content of the composite hyaluronic acid or a salt thereof is 1-15 wt %; preferably the content of the yeast fermentation product filtrate or lysate is 5-30 wt %, and the content of the composite hyaluronic acid or a salt thereof is 1-10 wt %.

3. The moisture-locking and repairing composition for hair according to claim 1, wherein the ratio of content of the yeast fermentation product filtrate or lysate to the composite hyaluronic acid or a salt thereof is 1:4 to 9:1, preferably 1:3 to 2:1.

4. The moisture-locking and repairing composition for hair according to claim 1, wherein the composite hyaluronic acid or a salt thereof is a composition of oligomeric hyaluronic acid or a salt thereof and low molecular weight hyaluronic acid or a salt thereof, and the mass ratio of the oligomeric hyaluronic acid or a salt thereof to the low molecular weight hyaluronic acid or a salt thereof is 1:5 to 5:1, preferably 1:4 to 3:2.

5. The moisture-locking and repairing composition for hair according to claim 4, wherein the molecular weight of the oligomeric hyaluronic acid or a salt thereof is less than or equal to 8 kDa, preferably less than or equal to 5 kDa; and the molecular weight of the low molecular weight hyaluronic acid or a salt thereof is 20-80 kDa, preferably 30-50 kDa.

6. The moisture-locking and repairing composition for hair according to claim 4, wherein a salt of the oligomeric hyaluronic acid and a salt of the low molecular weight hyaluronic acid are both selected from a sodium salt, potassium salt, calcium salt, zinc salt or magnesium salt thereof.

7. The moisture-locking and repairing composition for hair according to claim 1, wherein the yeast fermentation product filtrate or lysate refers to a fermentation filtrate or lysate obtained by fermenting rice, bean or wheat and other plants with yeast; preferably the yeast fermentation product filtrate or lysate refers to the fermentation filtrate or lysate obtained by fermenting rice with yeast.

8. The moisture-locking and repairing composition for hair according to claim 1, wherein it further comprises water for making up the balance.

9. Use of the moisture-locking and repairing composition for hair according to claim 1 in a hair product, preferably the hair product is a rinse-off or leave-on hair care product, or a rinse-off or leave-on hair cleansing product.

10. A hair product, wherein its functional components comprise the moisture-locking and repairing composition for hair according to claim 1.

11. The hair product according to claim 10, wherein the content of the moisture-locking and repairing composition for hair in the hair product is 0.5-10%, preferably 1-5%, and more preferably 2-3%; preferably, the hair product is a rinse-off and/or leave-on hair care product, or a rinse-off and/or leave-on hair cleansing product.
